(19) United States
(12) Patent Application Publication
(10) Pub. No.: US 2024/0174656 A1
(43) Pub. Date: May 30, 2024

(54) FUSED RING AROMATIC HYDROCARBON DERIVATIVE, PREPARATION METHOD THEREFOR, AND USE THEREOF IN LITHOGRAPHY

(71) Applicant: INSTITUTE OF CHEMISTRY, CHINESE ACADEMY OF SCIENCES, Beijing (CN)

(72) Inventors: Guoqiang YANG, Beijing (CN); Xue CONG, Beijing (CN); Xudong GUO, Beijing (CN); Rui HU, Beijing (CN); Shuangqing WANG, Beijing (CN)

(21) Appl. No.: 18/503,539

(22) Filed: Nov. 7, 2023

(30) Foreign Application Priority Data
Nov. 10, 2022 (CN) 202211406736.X

(51) Int. Cl. C07D 407/14; G03F 7/00; G03F 7/031

(52) U.S. Cl. CPC C07D 407/14; G03F 7/031; G03F 7/70033

(57) ABSTRACT

A negative chemically amplified photoresist of a fused ring aromatic hydrocarbon derivative, a preparation method therefor, and use thereof are provided. A matrix component in the photoresist is a fused ring aromatic hydrocarbon derivative represented by general formula (I) that can be dissolved in organic solvents commonly used in photoresists. The photoresist composition of the present disclosure can be prepared to give a uniform film, and a molecular glass serving as the matrix component is not precipitated in the film preparation process.

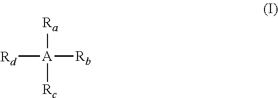

### TECHNICAL FIELD

The present disclosure belongs to the technical field of photoresists, and particularly relates to a fused ring aromatic hydrocarbon derivative, a preparation method therefor, and use thereof in lithography.

### BACKGROUND

In the manufacture of integrated circuits, lithography technology is a very critical process, which utilizes chemical reactions of photoresists during exposure to transfer a circuit pattern on a mask to a photoresist film, and then transfers the pattern to a silicon substrate after the subsequent etching process. Therefore, photoresists are the most important materials in the lithography and etching process, and the resolution of photoresists determines the critical dimension and integration level of integrated circuits.

The photoresist is composed of a host material, a photosensitizer (a photoacid generator), a solvent, and an additive. With the continuous development of integrated circuits, the performance requirements of the photoresist are higher and higher. The resolution, sensitivity, and line edge roughness or line width roughness of the photoresist are three important parameters for the evaluation of the photoresist performance. These three parameters restrict each other. Currently, researchers are working on how to coordinate the relationship among the three parameters to design better photoresist materials. A chemically amplified photoresist enables acid-sensitive groups in the material to perform deprotection or crosslinking reactions through acids generated by decomposing a photoacid generator, and simultaneously release new acids, thereby playing the effect of greatly improving the sensitivity of the photoresist. The traditional high-molecular chemically amplified photoresist causes problems of low resolution and poor line edge roughness of the photoresist due to large molecular weight and uneven distribution. The host material of the molecular glass photoresist is a small molecular compound, has relatively high glass transition temperature, integrates the advantages of polymers and small molecules, has small molecular weight and monodispersity, and can meet the requirements of lithography after the introduction of an acid-sensitive group.

A fused ring aromatic hydrocarbon is an organic substance formed by fusing two or more benzene rings sharing two adjacent carbon atoms, such as naphthalene, anthracene, phenanthrene, and pyrene. The compound has the characteristics of high melting point and high boiling point, and is more suitable for being a core structure of the molecular glass photoresist due to the conjugation of a plurality of benzene rings. The molecular glass photoresist taking the fused ring aromatic hydrocarbon as the center has a relatively high melting point and thermal stability as well as more ideal etching resistance. The present disclosure develops a novel molecular glass photoresist taking a fused ring aromatic hydrocarbon as a center, which is expected to achieve a negative chemically amplified photoresist with higher resolution and contrast as well as high etching resistance.

### SUMMARY

The object of the present disclosure is to provide a fused ring aromatic hydrocarbon derivative and a preparation method therefor.

Another object of the present disclosure is to provide use of a plurality of fused ring aromatic hydrocarbon derivatives described above in lithography and a negative photoresist composition. The present disclosure provides a compound represented by formula (I):

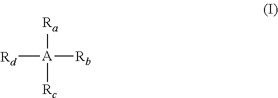

wherein A is selected from a fused ring aromatic hydrocarbon;
Rₐ, R_{b}, R_{c}, and R_{d} are the same or different and are each independently selected from H or

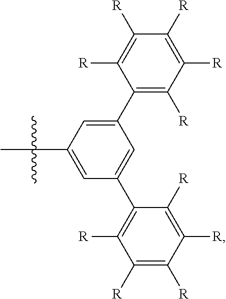

provided that at least one of Rₐ, R_{b}, R_{c}, and R_{d} is

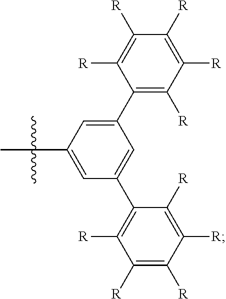

each R is the same or different and is independently selected from H, —OC₁₋₂₀ alkyl, or OR₁, provided that at least one R is OR₁ or H; R₁ is selected from the following groups that are unsubstituted or optionally substituted with one, two, or more R₁₁: C₂₋₂₀ alkenyl and 3- to 20-membered epoxy; each R₁₁ is the same or different and is independently selected from oxo(═O) and the following groups that are unsubstituted or optionally substituted with one, two, or more R₁₂: C₁₋₂₀ alkyl, C₁₋₂₀ alkoxy, C₂₋₂₀ alkenyl, 3- to 20-membered heterocyclyl, and C₆₋₂₀ aryl; each R₁₂ is the same or different and is independently selected from C₁₋₂₀ alkyl, C₂₋₂₀ alkenyl, C₁₋₂₀ alkoxy, and C₆₋₂₀ aryl.

According to an embodiment of the present disclosure, the fused ring aromatic hydrocarbon is selected from C₉₋₄₀ aromatic hydrocarbon, preferably C₁₀₋₁₆ aromatic hydrocarbon.

According to an embodiment of the present disclosure, the fused ring aromatic hydrocarbon is selected from naphthalene, anthracene, phenanthrene, or pyrene.

According to an embodiment of the present disclosure, Rₐ, R_{b}, R_{c}, and R_{d} are the same or different and are each independently selected from H or

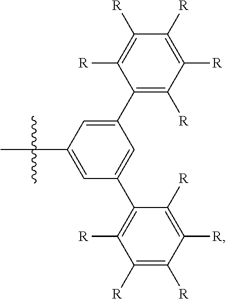

provided that one, two, three, or four of Rₐ, R_{b}, R_{c}, and R_{d} are

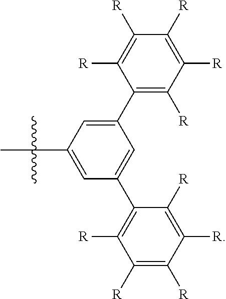

According to an embodiment of the present disclosure, Rₐ, R_{b}, R_{c}, and R_{d} are selected from

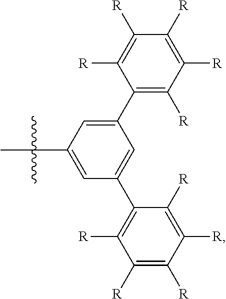

wherein in the

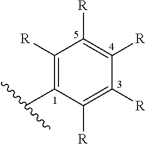

group, when there is only one R, it is preferably linked at position 4; when there are two R, they are preferably linked at positions 3 and 4, or positions 4 and 5; when there are three R, they are preferably linked at positions 3, 4, and 5.

According to an embodiment of the present disclosure, each R is the same or different and is independently selected from H or OR₁, provided that R is not all H; R₁ is selected from the following groups that are unsubstituted or optionally substituted with one, two, or more R₁₁: C₁₋₆ alkyl and 3- to 8-membered heterocyclyl; each R₁₁ is the same or different and is independently selected from oxo(═O) and the following groups that are unsubstituted or optionally substituted with one, two, or more R₁₂: C₁₋₆ alkyl, C₂₋₆ alkenyl, and oxygen-containing 3- to 8-membered heterocyclyl.

According to an embodiment of the present disclosure, each R is the same or different and is independently selected from H or OR₁, provided that R is not all H; R₁ is selected from the following groups that are unsubstituted or optionally substituted with one, two, or more R₁₁: oxygen-containing 3- to 8-membered heterocyclyl, C₂₋₆ alkenyl-C₁₋₆ alkyl, and oxygen-containing 3- to 8-membered heterocyclyl-C₁₋₆ alkyl; each R₁₁ is the same or different and is independently selected from oxo(═O) and C₁₋₆ alkyl.

According to an embodiment of the present disclosure, each R is the same or different and is independently selected from H or OR₁, provided that R is not all H; R₁ is selected from

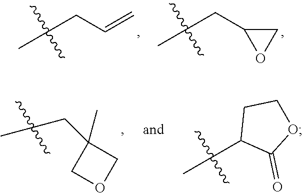

the “ [symbol] ” is a linking site.

According to an embodiment of the present disclosure, the compound represented by formula (I) preferably has a structure represented by formula (A) or formula (B):

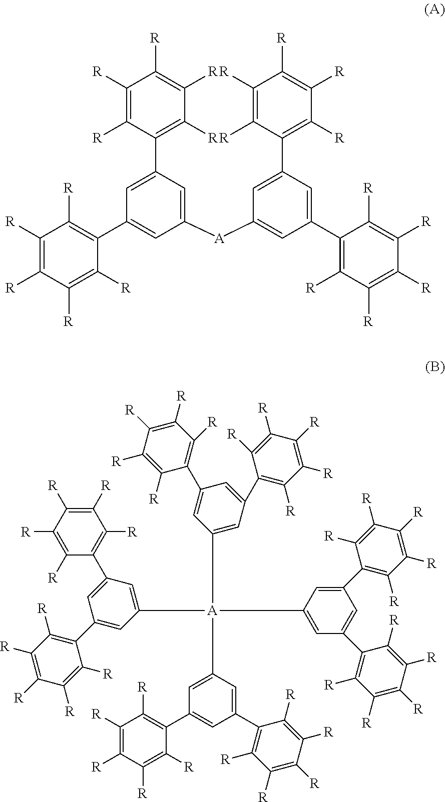

wherein R and A have the definitions described above.

According to a preferred embodiment of the present disclosure, the compound represented by formula (I) is selected from the following structures:

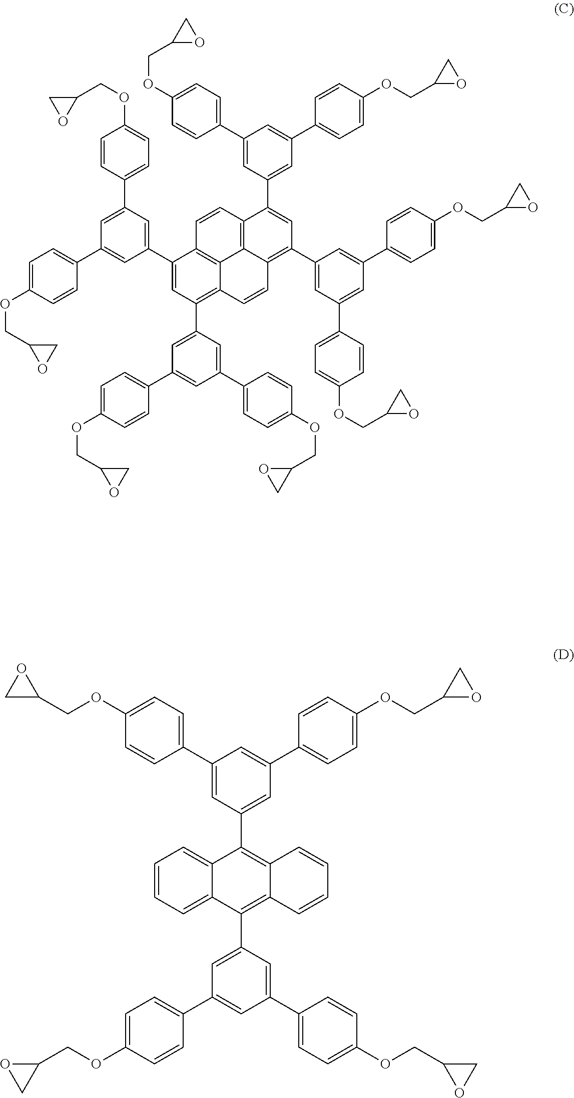 [SMILES: c1cc(-c2cc(-c3ccc(OCC4CO4)cc3)cc(-c3cc(-c4cc(-c5ccc(OCC6CO6)cc5)cc(-c5ccc(OCC6CO6)cc5)c4)c4ccc5c(-c6cc(-c7ccc(OCC8CO8)cc7)cc(-c7ccc(OCC8CO8)cc7)c6)cc(-c6cc(-c7ccc(OCC8CO8)cc7)cc(-c7ccc(OCC8CO8)cc7)c6)c6ccc3c4c65)c2)ccc1OCC1CO1 ; c1ccc2c(-c3cc(-c4ccc(OCC5CO5)cc4)cc(-c4ccc(OCC5CO5)cc4)c3)c3ccccc3c(-c3cc(-c4ccc(OCC5CO5)cc4)cc(-c4ccc(OCC5CO5)cc4)c3)c2c1]

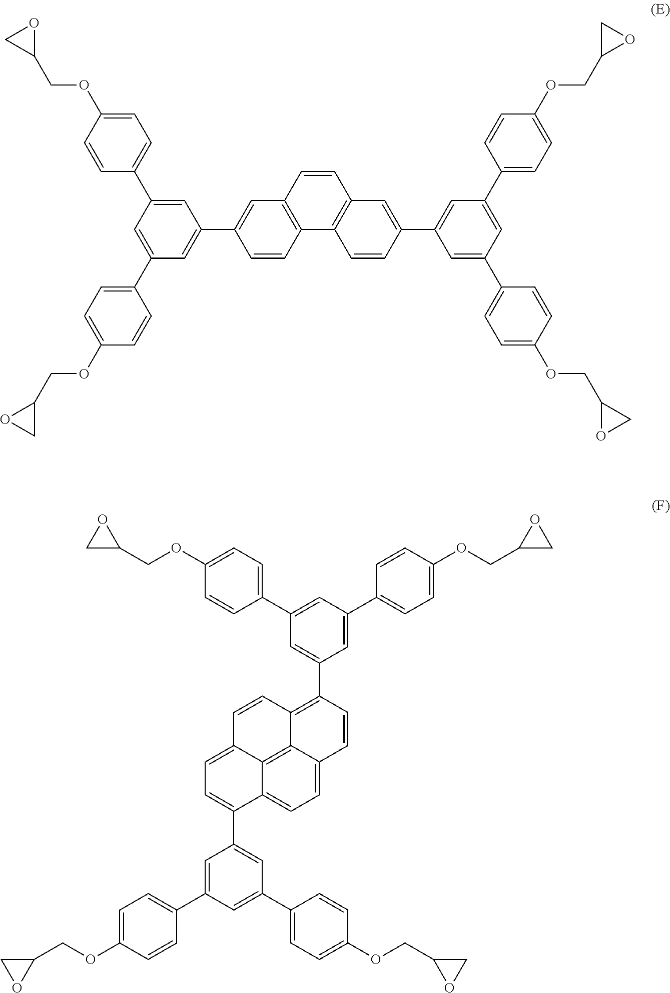 [SMILES: c1cc(-c2cc(-c3ccc(OCC4CO4)cc3)cc(-c3ccc4c(ccc5cc(-c6cc(-c7ccc(OCC8CO8)cc7)cc(-c7ccc(OCC8CO8)cc7)c6)ccc54)c3)c2)ccc1OCC1CO1 ; c1cc(-c2cc(-c3ccc(OCC4CO4)cc3)cc(-c3ccc4ccc5c(-c6cc(-c7ccc(OCC8CO8)cc7)cc(-c7ccc(OCC8CO8)cc7)c6)ccc6ccc3c4c65)c2)ccc1OCC1CO1]

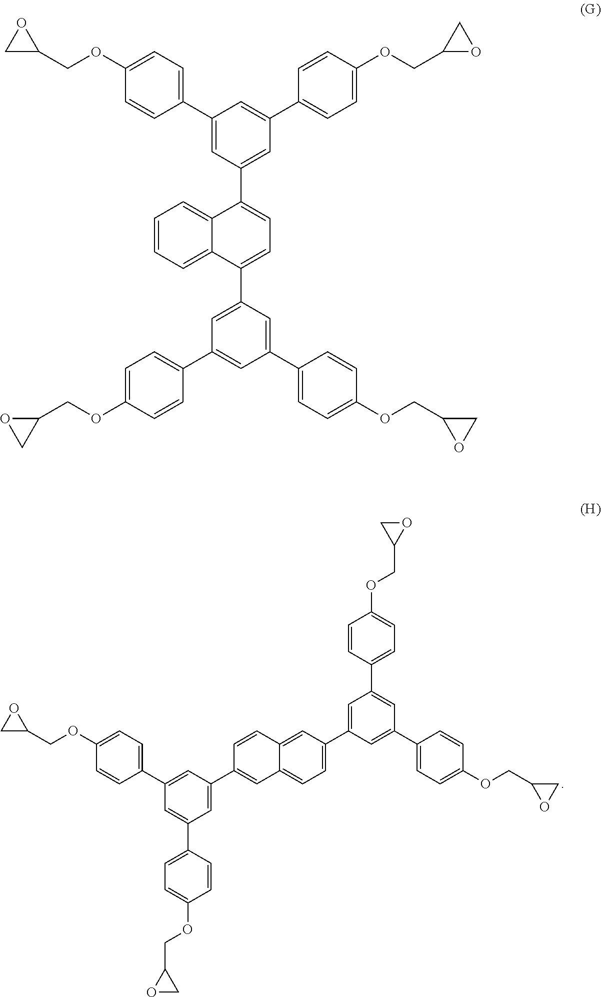 [SMILES: c1ccc2c(-c3cc(-c4ccc(OCC5CO5)cc4)cc(-c4ccc(OCC5CO5)cc4)c3)ccc(-c3cc(-c4ccc(OCC5CO5)cc4)cc(-c4ccc(OCC5CO5)cc4)c3)c2c1 ; c1cc(-c2cc(-c3ccc(OCC4CO4)cc3)cc(-c3ccc4cc(-c5cc(-c6ccc(OCC7CO7)cc6)cc(-c6ccc(OCC7CO7)cc6)c5)ccc4c3)c2)ccc1OCC1CO1]

The present disclosure further provides a preparation method for the compound represented by formula (I), comprising the following steps:

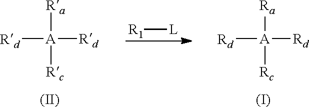

reacting a compound represented by formula (II) with R₁-L to give the compound represented by formula (I);
wherein A, Rₐ, R_{b}, R_{c}, R_{d}, and R₁ have the definitions described above;
R′ₐ, R′_{b}, R′_{c}, and R′_{d} are

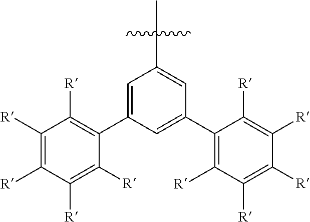

or H, provided that not all are H; each R′ is the same or different and is independently selected from OH or H, provided that not all are H; L is selected from a leaving group such as halogen or p-toluenesulfonate.

According to an embodiment of the present disclosure, the compound (I) represented by formula (I) is obtained by introducing R₁ groups on the basis of the compound represented by formula (II) for complete or partial protection.

According to an embodiment of the present disclosure, R₁-L is selected from 2-(bromomethyl)oxirane, allyl bromide, α-bromo-γ-butyrolactone, or 3-methyl-3-(tosyloxymethyl)oxetane.

According to an embodiment of the present disclosure, L is selected from bromine.

According to an embodiment of the present disclosure, in the method described above, the reaction is performed in an organic solvent, wherein the organic solvent is selected from formamide, chloroform, DMF, acetonitrile, tetrahydrofuran, N-methylpyrrolidone, and the like, preferably N-methylpyrrolidone.

According to an embodiment of the present disclosure, in the method described above, the reaction is performed in the presence of an alkaline compound, wherein the alkaline compound is selected from Na₂CO₃, K₂CO₃, NaHCO₃, C_{S2}CO₃, and the like.

According to an embodiment of the present disclosure, in the method described above, the reaction is performed at a temperature of 30-80° C., preferably 60-70° C.; the reaction is performed for 12-36 h, preferably 18-24 h.

The present disclosure further provides use of the compound represented by formula (I) in lithography, such as use thereof in a photoresist, preferably use thereof in the preparation of a negative photoresist. The present disclosure further provides a negative photoresist composition comprising a matrix, wherein the matrix is selected from at least one of compounds represented by formula (I).

According to an embodiment of the present disclosure, the composition further comprises a photoacid generator, wherein the photoacid generator is, for example, selected from ionic or non-ionic acid generators, such as at least one of triphenylsulfonium triflate, triphenylsulfonium nonaflate, bis(4-tert-butylphenyl)iodonium p-toluenesulfonate, N-hydroxynaphthalimide triflate, and benzyl(4-hydroxyphenyl)methylsulfonium hexafluoroantimonate.

According to an embodiment of the present disclosure, the composition further comprises an organic solvent. The organic solvent is, for example, selected from alkane, ester, ether, and haloalkane compounds. The preferred organic solvent is at least one of 1,2,3-trichloropropane, anisole, propylene glycol methyl ether acetate, propylene glycol monoacetate, propylene glycol diacetate, ethyl lactate, propylene glycol monomethyl ether, methyl ethyl ketone, methyl isobutyl ketone, neopentyl acetate, butyl acetate, diethylene glycol ethyl ether, dichloromethane, and tetrahydrofuran.

According to an embodiment of the present disclosure, in the photoresist composition, the mass of the matrix accounts for 2%-30%, preferably 4%-20%, of the total mass of the negative photoresist composition.

According to an embodiment of the present disclosure, in the photoresist composition, the mass of the photoacid generator accounts for 2%-30%, preferably 5%-20%, of the mass of the matrix.

According to an embodiment of the present disclosure, in the photoresist composition, the mass of the organic solvent accounts for 70%-96% of the total mass of the photoresist composition.

According to an embodiment of the present disclosure, the photoresist composition further comprises other additives, such as sensitizers, surfactants, dyes, stabilizers, and the like.

The present disclosure further provides use of the photoresist composition in ultraviolet (365 nm) lithography, deep ultraviolet (248 nm and 193 nm) lithography, extreme ultraviolet (13.5 nm, EUV) lithography, and electron beam lithography (EBL).

### Beneficial Effects

The matrix component in the negative photoresist composition of the present disclosure takes a fused ring aromatic hydrocarbon represented by formula (I) as a central core structure, so the negative photoresist composition has a relatively high melting point, can meet the requirements of lithography technology, has a stable structure, and has no change of a film structure in high-temperature baking. The negative molecular glass photoresist provided by the present disclosure is an amorphous small molecular compound, has the advantages of relatively good film-forming property, relatively high thermal stability, resulting in less proneness to deformation during storage, and low viscosity without the use of additional solvents for dilution. After exposure, the exposed pattern has excellent resolution and relatively good sensitivity.

### BRIEF DESCRIPTION OF THE DRAWINGS

FIG. 1 is a diagram showing a thermogravimetric analysis test of compound C prepared in Example 1.

FIG. 2 is a diagram showing an X-ray diffraction test of the compound C prepared in Example 1.

FIG. 3 is an electron micrograph of an electron beam lithographic pattern of a photoresist composition prepared in Example 3.

FIG. 4 is an electron micrograph of an electron beam lithographic pattern of the photoresist composition prepared in Example 3.

FIG. 5 is an electron micrograph of an electron beam lithographic pattern of a photoresist composition prepared in Example 4.

### DEFINITIONS AND DESCRIPTION

Unless otherwise defined, all technical and scientific terms used herein have the same meaning as commonly understood by those skilled in the art to which the subject matter of the claims belong.

The term “halogen” includes F, Cl, Br, or I.

The term “C₁₋₂₀ alkyl” should be understood to refer to a linear or branched saturated monovalent hydrocarbyl group having 1-20 carbon atoms, preferably “C₁₋₆ alkyl”. “C₁₋₆ alkyl” refers to linear and branched alkyl groups having 1, 2, 3, 4, 5, or 6 carbon atoms. The alkyl is, for example, methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl, etc., or isomers thereof.

The term “C₂₋₂₀ alkenyl” should be understood to refer to a linear or branched monovalent hydrocarbyl group containing one or more double bonds and having 2-20 carbon atoms, preferably “C₂₋₁₂ alkenyl”. “C₂₋₁₂ alkenyl” should be understood to preferably refer to a linear or branched monovalent hydrocarbyl group containing one or more double bonds and having 2, 3, 4, 5, 6, 7, 8, 9, 10, 11, or 12 carbon atoms, more preferably “C₂₋₈ alkenyl”. “C₂₋₈ alkenyl” should be understood to preferably refer to a linear or branched monovalent hydrocarbyl group containing one or more double bonds and having 2, 3, 4, 5, 6, 7, or 8 carbon atoms, for example, having 2, 3, 4, 5, or 6 carbon atoms (i.e., C₂₋₆ alkenyl) or having 2 or 3 carbon atoms (i.e., C₂₋₃ alkenyl). It should be understood that in the case that the alkenyl comprises more than one double bond, the double bonds can be separated from one another or conjugated. The alkenyl is, for example, vinyl, allyl, (E)-2-methylvinyl, (Z)-2-methylvinyl, (E)-but-2-enyl, (Z)-but-2-enyl, (E)-but-1-enyl, (Z)-but-1-enyl, pent-4-enyl, (E)-pent-3-enyl, (Z)-pent-3-enyl, (E)-pent-2-enyl, (Z)-pent-2-enyl, (E)-pent-1-enyl, (Z)-pent-1-enyl, hex-5-enyl, (E)-hex-4-enyl, (Z)-hex-4-enyl, (E)-hex-3-enyl, (Z)-hex-3-enyl, (E)-hex-2-enyl, (Z)-hex-2-enyl, (E)-hex-1-enyl, (Z)-hex-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, 3-methylbut-3-enyl, 2-methylbut-3-enyl, 1-methylbut-3-enyl, 3-methylbut-2-enyl, (E)-2-methylbut-2-enyl, (Z)-2-methylbut-2-enyl, (E)-1-methylbut-2-enyl, (Z)-1-methylbut-2-enyl, (E)-3-methylbut-1-enyl, (Z)-3-methylbut-1-enyl, (E)-2-methylbut-1-enyl, (Z)-2-methylbut-1-enyl, (E)-1-methylbut-1-enyl, (Z)-1-methylbut-1-enyl, 1,1-dimethylprop-2-enyl, 1-ethylprop-1-enyl, 1-propylvinyl, or 1-isopropylvinyl.

The term “3- to 20-membered heterocyclyl” refers to a saturated or unsaturated non-aromatic ring or ring system; for example, it is a 4-, 5-, 6-, or 7-membered monocyclic ring system, a 7-, 8-, 9-, 10-, 11-, or 12-membered bicyclic (e.g., fused, bridged, or spiro) ring system, or a 10-, 11-, 12-, 13-, 14-, or 15-membered tricyclic ring system, and contains at least one, e.g., 1, 2, 3, 4, 5, or more heteroatoms selected from O, S, and N, wherein N and S may also be optionally oxidized to various oxidized forms to form nitrogen oxides,—S(O)—or —S(O)₂—. Preferably, the heterocyclyl may be selected from “3- to 10-membered heterocyclyl”. The term “3- to 10-membered heterocyclyl” refers to a saturated or unsaturated non-aromatic ring or ring system and contains at least one heteroatom selected from O, S, and N. The heterocyclyl may be connected to the rest of the molecule through any one of the carbon atoms or the nitrogen atom (if present). The heterocyclyl may include fused or bridged rings as well as spiro rings. In particular, the heterocyclyl may include, but is not limited to: 4-membered rings such as azetidinyl and oxetanyl; 5-membered rings such as tetrahydrofuranyl, dioxolyl, pyrrolidinyl, imidazolidinyl, pyrazolidinyl, and pyrrolinyl; 6-membered rings such as tetrahydropyranyl, piperidyl, morpholinyl, dithianyl, thiomorpholinyl, piperazinyl, and trithianyl; or 7-membered rings such as diazepanyl. Optionally, the heterocyclyl may be benzo-fused.

The term “C₉₋₄₀ aromatic hydrocarbon” should be understood to preferably refer to an aromatic or partially aromatic fused ring having 9-40 carbon atoms, which may be a single aromatic ring or multiple aromatic rings fused together, preferably “C₉₋₂₀ aromatic hydrocarbon”. The term “C₉₋₂₀ aromatic hydrocarbon” should be understood to preferably refer to an aromatic or partly aromatic fused ring having 9, 10, 11, 12, 13, 14, 15, 16, 17, 18, 19, or 20 carbon atoms, particularly a ring having 10-16 carbon atoms (“C₁₀₋₁₆ aromatic hydrocarbon”), for example, a ring having 9 carbon atoms (“C₉ aromatic hydrocarbon”) such as indan or indene, or a ring having 10 carbon atoms (“C₁₀ aromatic hydrocarbon”) such as tetrahydronaphthalene, dihydronaphthalene, or naphthalene, or a ring having 13 carbon atoms (“C₁₃ aromatic hydrocarbon”) such as fluorene, or a ring having 14 carbon atoms (“C₁₄ aromatic hydrocarbon”) such as anthracene, or a ring having 16 carbon atoms (“C₁₆ aromatic hydrocarbon”) such as pyrene. When the C₉₋₄₀ aromatic hydrocarbon is substituted, it may be monosubstituted or polysubstituted. In addition, the substitution site is not limited, and may be, for example, ortho-substitution, para-substitution, or meta-substitution.

### DETAILED DESCRIPTION

The embodiments of the present disclosure will be further illustrated in detail with reference to the following specific examples. It will be appreciated that the following examples are merely exemplary illustrations and explanations of the present disclosure, and should not be construed as limiting the protection scope of the present disclosure. All techniques implemented based on the content of the present disclosure described above are included within the protection scope of the present disclosure. Unless otherwise stated, the starting materials and reagents used in the following examples are all commercially available products or can be prepared using known methods.

### Example 1: Preparation of Compound C

Synthetic Route of compound C:

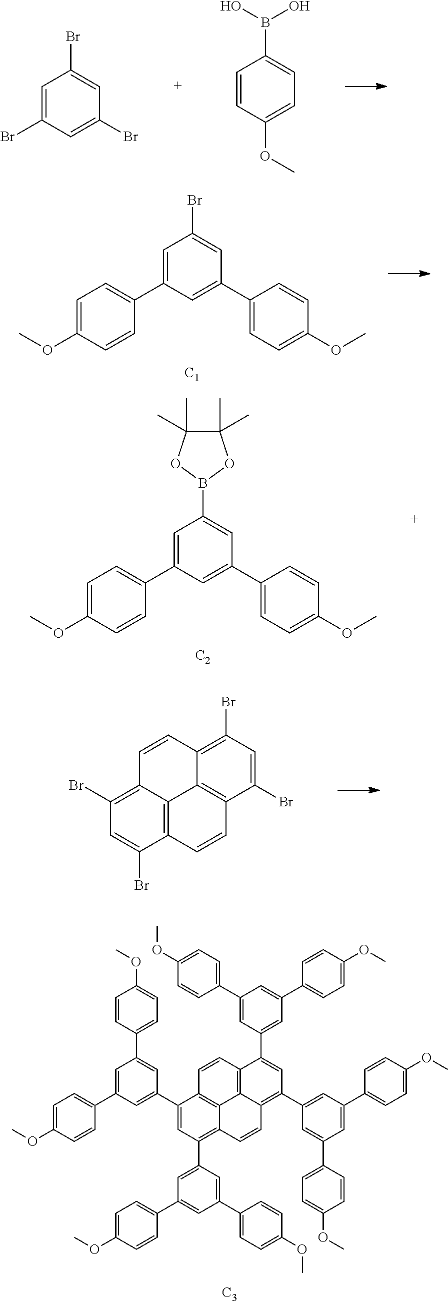

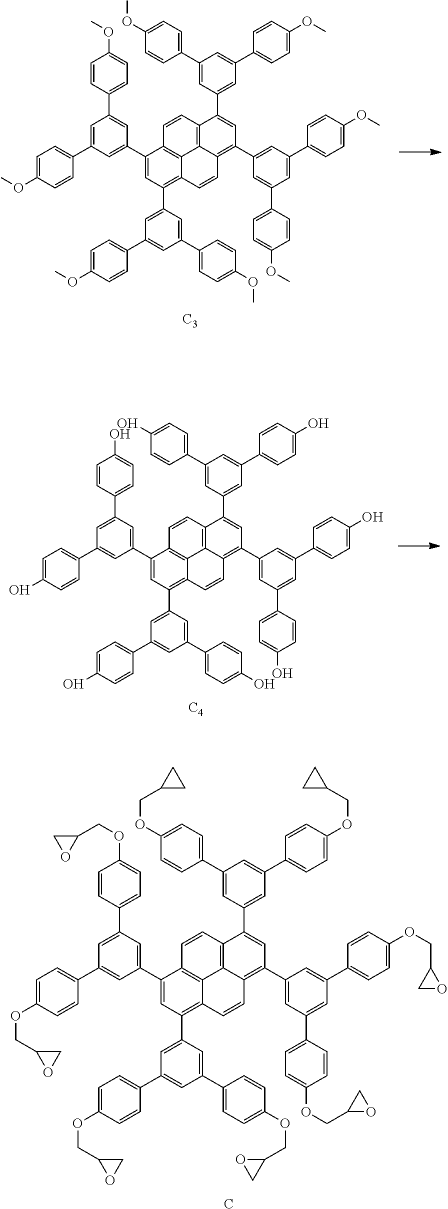

### Synthesis of Compound C₁:

15.2 g of 4-methoxyphenylboronic acid, 0.92 g of tetrakis(triphenylphosphine)palladium(0), 17 g of Na₂CO₃, and a magnetic stir bar were placed in a three-necked flask (the three-necked flask was connected to a gas-guide tube, a constant pressure dropping funnel, and a rubber septum, respectively). The reaction system was vacuumized and filled with argon three times, thus enabling the reaction to be performed in an argon atmosphere. 150 mL of 1,4-dioxane and 100 mL of ultrapure water were injected into the three-necked flask. 12.6 g of 1,3,5-tribromobenzene was dissolved in 50 mL of 1,4-dioxane, and the solution was injected into the dropping funnel. The reaction system was heated to 80° C. and stirred, and the solution in the dropping funnel was added dropwise. After the dropwise addition was completed, the system was heated to 100° C. and stirred for 24 h. After the reaction was completed, the reaction liquid was washed with a large amount of saturated brine and dichloromethane, dried over anhydrous Na₂SO₄ for 1 h, and filtered to give a filtrate. The filtrate was concentrated by rotary evaporation and separated by column chromatography to give compound C₁ (6.49 g).

### Synthesis of Compound C₂:

4.57 g of bis(pinacolato)diboron, 4.42 g of KOAc, 327 mg of Pd(dppf)Cl₂, and a magnetic stir bar were placed in a three-necked flask (the three-necked flask was connected to a gas-guide tube, a constant pressure dropping funnel, and a rubber septum, respectively). The reaction system was vacuumized and filled with argon three times, thus enabling the reaction to be performed in an argon atmosphere. 35 mL of 1,4-dioxane was injected into the three-necked flask. 5.54 g of compound C₁ was dissolved in 30 mL of 1,4-dioxane, and the solution was injected into the dropping funnel. The reaction system was heated to 80° C. and stirred, the solution in the dropping funnel was added dropwise, and the reaction mixture was reacted for 24 h. After the reaction was completed, the reaction liquid was washed with a large amount of saturated brine and dichloromethane, dried over anhydrous Na₂SO₄ for 1 h, and filtered to give a filtrate. The filtrate was concentrated by rotary evaporation, a large amount of n-hexane was added for precipitation, sonication was performed, and the reaction liquid was filtered to give compound C₂ (5.71 g).

### Synthesis of Compound C₃:

1.04 g of 1,3,6,8-tetrabromopyrene, 2.21 g of Na₂CO₃, 0.277 g of tetrakis(triphenylphosphine)palladium(0), and a magnetic stir bar were placed in a three-necked flask (the three-necked flask was connected to a gas-guide tube, a constant pressure dropping funnel, and a rubber septum, respectively). The reaction system was vacuumized and filled with argon three times, thus enabling the reaction to be performed in an argon atmosphere. 10 mL of 1,4-dioxane and 8 mL of ultrapure water were injected into the three-necked flask. 4.16 g of the compound C₂ was dissolved in 40 mL of 1,4-dioxane, and the solution was injected into the dropping funnel. The reaction system was heated to 80° C. and stirred, and the solution in the dropping funnel was added dropwise. After the dropwise addition was completed, the system was heated to 100° C. and stirred for 24 h. After the reaction was completed, the reaction liquid was washed with a large amount of saturated brine and dichloromethane, dried over anhydrous Na₂SO₄ for 1 h, and filtered to give a filtrate. The filtrate was concentrated by rotary evaporation and separated by column chromatography to give compound C₃ (1.63 g).

### Synthesis of Compound C₄:

1.36 g of the compound C₃ was dissolved in 70 mL of dichloromethane, and then the mixture was added to a three-necked flask with a magnetic stir bar (the three-necked flask was connected to a gas-guide tube, a constant pressure dropping funnel, and a rubber septum, respectively). The system was placed in an ice-water bath, and 4.64 mL of BBr₃ was injected into the dropping funnel. The reaction system was stirred. After the slow dropwise addition was completed, the reaction system was cooled to room temperature and reacted for 24 h. After the reaction was completed, the reaction liquid was transferred into another constant pressure dropping funnel connected to a three-necked flask. 100 mL of ice water was added to the three-necked flask, and the reaction liquid was slowly added dropwise in an ice-water bath and stirred for 2 h. After the reaction was completed, the reaction liquid was washed with a large amount of saturated brine and ethyl acetate, dried over anhydrous Na₂SO₄ for 1 h, and filtered to give a filtrate. The filtrate was concentrated by rotary evaporation, a large amount of n-hexane was added for precipitation, sonication was performed, and the reaction liquid was filtered to give compound C₄ (1.24 g).

### Synthesis of Compound C:

673 mg of KOH and 1 mL of 2-(bromomethyl)oxirane were placed in a two-necked flask (the two-necked flask was connected to a constant pressure dropping funnel and a rubber septum, respectively). 5 mL of NMP was injected into the two-necked flask. 1.24 g of the compound C₄ was dissolved in 10 mL of NMP, and the solution was injected into the dropping funnel. The reaction system was stirred at 60-70° C., and the solution in the dropping funnel was added dropwise. After the dropwise addition was completed, the reaction mixture was reacted for 24 h. After the reaction was completed, the reaction mixture was diluted with dichloromethane, washed with a large amount of saturated brine, dried over anhydrous Na₂SO₄ for 1 h, and filtered to give a filtrate. The filtrate was concentrated by rotary evaporation and separated by column chromatography to give compound C (720 mg).

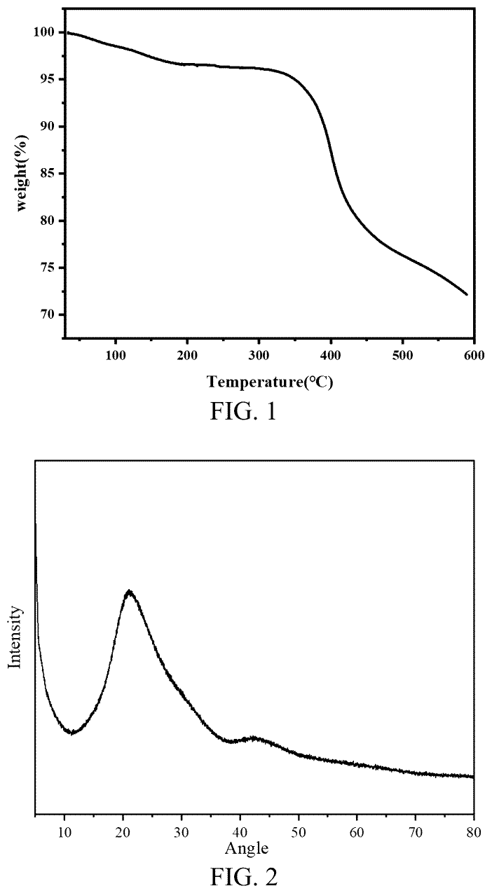

A thermogravimetric analysis of the compound C is shown in FIG. 1, and the decomposition temperature is about 350° C.; an XRD analysis is shown in FIG. 2, indicating that the compound C is an amorphous compound and does not crystallize.

¹H NMR (300 MHz, CDCl₃) δ 8.35 (s, 4H), 8.21 (s, 2H), 7.81 (s, 12H), 7.66 (d, J=8.6 Hz, 16H), 7.01 (d, J=8.5 Hz, 16H), 4.27 (dd, J=10.9, 2.9 Hz, 8H), 4.00 (dd, J=10.9, 5.7 Hz, 8H), 3.38 (s, 8H), 2.92 (t, J=4.4 Hz, 8H), 2.78 (s,8H). HRMS(MALDI): calculated [M+H]⁺: 1691.63; found: 1691.62.

### Example 2: Preparation of Compound F

### Synthetic Route of Compound F:

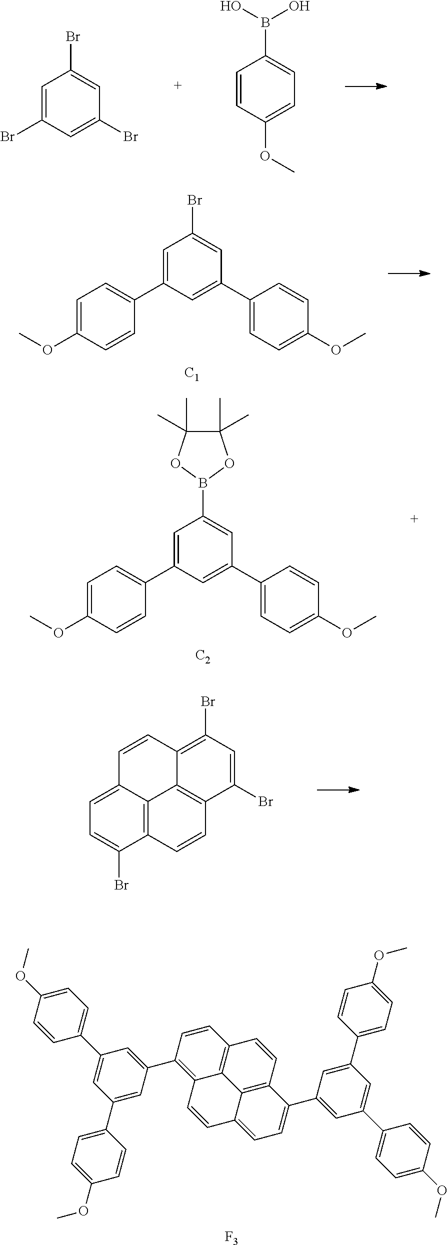

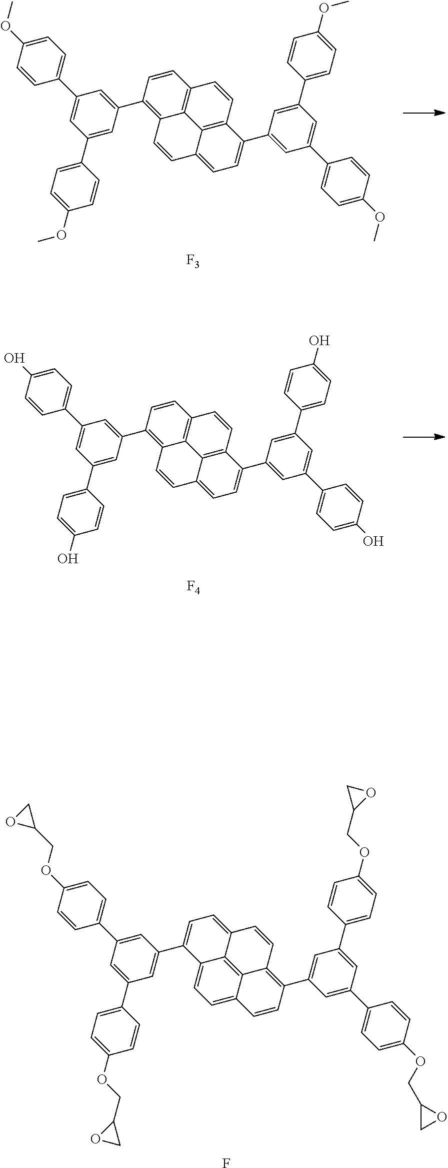

### Synthesis of Compound F₃:

1.08 g of 1,6-dibromopyrene, 2.86 g of Na₂CO₃, 0.208 g of tetrakis(triphenylphosphine)palladium(0), and a magnetic stir bar were placed in a three-necked flask (the three-necked flask was connected to a gas-guide tube, a constant pressure dropping funnel, and a rubber septum, respectively). The reaction system was vacuumized and filled with argon three times, thus enabling the reaction to be performed in an argon atmosphere. 10 mL of 1,4-dioxane and 15 mL of ultrapure water were injected into the three-necked flask. 3.12 g of the compound C₂ was dissolved in 20 mL of 1,4-dioxane, and the solution was injected into the dropping funnel. The reaction system was heated to 80° C. and stirred, and the solution in the dropping funnel was added dropwise. After the dropwise addition was completed, the system was heated to 100° C. and stirred for 24 h. After the reaction was completed, the reaction liquid was washed with a large amount of saturated brine and dichloromethane, dried over anhydrous Na₂SO₄ for 1 h, and filtered to give a filtrate. The filtrate was concentrated by rotary evaporation, a large amount of petroleum ether was added for precipitation, sonication was performed, and the reaction liquid was filtered to give compound F₃ (2.34 g).

### Synthesis of Compound F₄:

2.34 g of the compound F₃ was dissolved in 30 mL of dichloromethane, and then the mixture was added to a three-necked flask with a magnetic stir bar (the three-necked flask was connected to a gas-guide tube, a constant pressure dropping funnel, and a rubber septum, respectively). The system was placed in an ice-water bath, and 1.74 mL of BBr₃ was injected into the dropping funnel. The reaction system was stirred. After the slow dropwise addition was completed, the reaction system was cooled to room temperature and reacted for 24 h. After the reaction was completed, the reaction liquid was transferred into another constant pressure dropping funnel connected to a three-necked flask. 50 mL of ice water was added to the three-necked flask, and the reaction liquid was slowly added dropwise in an ice-water bath and stirred for 2 h. After the reaction was completed, the reaction liquid was washed with a large amount of saturated brine and ethyl acetate, dried over anhydrous Na₂SO₄ for 1 h, and filtered to give a filtrate. The filtrate was concentrated by rotary evaporation, a large amount of n-hexane was added for precipitation, sonication was performed, and the reaction liquid was filtered to give compound F₄ (2.16 g).

### Synthesis of Compound F:

450 mg of KOH and 1 mL of 2-(bromomethyl)oxirane were placed in a two-necked flask (the two-necked flask was connected to a constant pressure dropping funnel and a rubber septum, respectively). 5 mL of NMP was injected into the two-necked flask. 722.84 mg of the compound F₄ was dissolved in 10 mL of NMP, and the solution was injected into the dropping funnel. The reaction system was stirred at 60-70° C., and the solution in the dropping funnel was added dropwise. After the dropwise addition was completed, the reaction mixture was reacted for 24 h. After the reaction was completed, the reaction mixture was diluted with dichloromethane, washed with a large amount of saturated brine, dried over anhydrous Na₂SO₄ for 1 h, and filtered to give a filtrate. The filtrate was concentrated by rotary evaporation and separated by column chromatography to give compound F (220 mg). ¹H NMR (300 MHz, CDCl3) δ 8.33 (d, J=9.4 Hz, 2H), 8.24 (d, J=7.9 Hz, 2H), 8.13-8.04 (m, 4H), 7.82 (d, J=21.6 Hz, 6H), 7.70 (d, J=8.5 Hz, 8H), 7.05 (d, J=8.5 Hz, 6H), 6.98 (s, 2H), 4.29 (d, J=8.1 Hz, 4H), 4.03 (dd, J=11.0, 5.6 Hz, 4H), 3.40 (s, 4H), 2.94 (t, J=4.4 Hz, 4H), 2.82 (d, J=14.1 Hz, 4H).

### Example 3: Photoresist Composition Comprising Compound C

### Photoresist Composition:

(1) matrix:

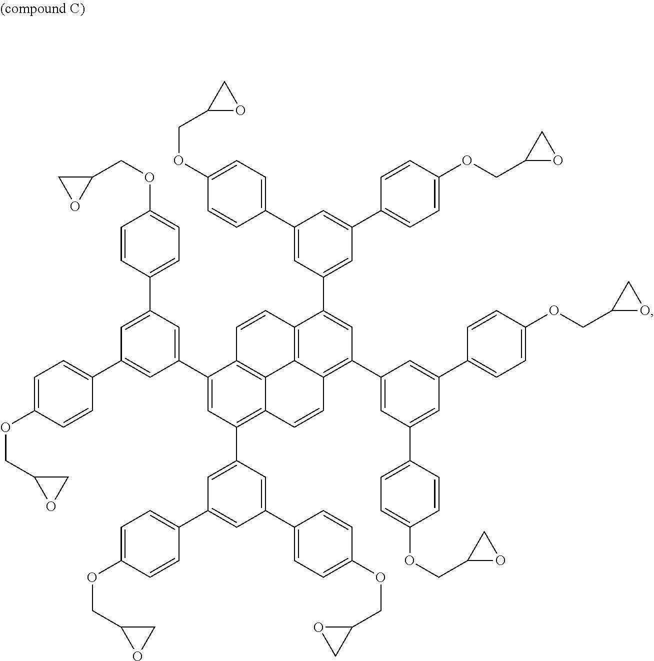 [SMILES: c1cc(-c2cc(-c3ccc(OCC4CO4)cc3)cc(-c3cc(-c4cc(-c5ccc(OCC6CO6)cc5)cc(-c5ccc(OCC6CO6)cc5)c4)c4ccc5c(-c6cc(-c7ccc(OCC8CO8)cc7)cc(-c7ccc(OCC8CO8)cc7)c6)cc(-c6cc(-c7ccc(OCC8CO8)cc7)cc(-c7ccc(OCC8CO8)cc7)c6)c6ccc3c4c65)c2)ccc1OCC1CO1]

50 mg;
(2) photoacid generator: benzyl(4-hydroxyphenyl)methylsulfonium hexafluoroantimonate, 3.75 mg; and
(3) organic solvent: propylene glycol methyl ether acetate (PGMEA), 2 mL.

### Example 4: Photoresist Composition Comprising Compound F

(1) matrix:

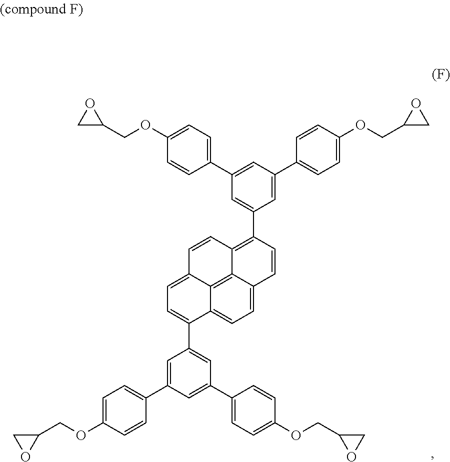 [SMILES: c1cc(-c2cc(-c3ccc(OCC4CO4)cc3)cc(-c3ccc4ccc5c(-c6cc(-c7ccc(OCC8CO8)cc7)cc(-c7ccc(OCC8CO8)cc7)c6)ccc6ccc3c4c65)c2)ccc1OCC1CO1]

50 mg;
(2) photoacid generator: benzyl(4-hydroxyphenyl)methylsulfonium hexafluoroantimonate, 3.75 mg; and
(3) organic solvent: propylene glycol methyl ether acetate (PGMEA), 2 mL.

### Example 5: Photoresist Composition Comprising Compound D

(1) matrix:

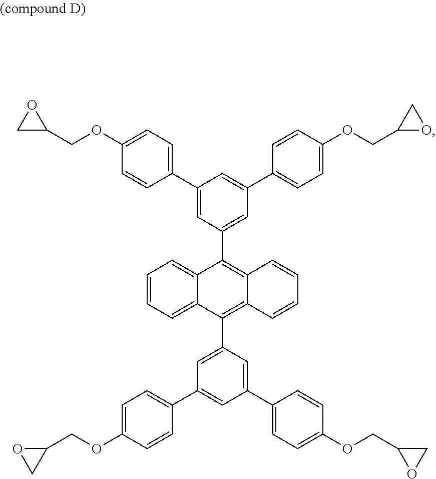 [SMILES: c1ccc2c(-c3cc(-c4ccc(OCC5CO5)cc4)cc(-c4ccc(OCC5CO5)cc4)c3)c3ccccc3c(-c3cc(-c4ccc(OCC5CO5)cc4)cc(-c4ccc(OCC5CO5)cc4)c3)c2c1]

50 mg;
(2) photoacid generator: benzyl(4-hydroxyphenyl)methylsulfonium hexafluoroantimonate, 2.5 mg; and
(3) organic solvent: propylene glycol methyl ether acetate (PGMEA), 2 mL.

Among them, the compound D was prepared by a method similar to that of Example 1 or 2. HRMS(MALDI) [M+H]⁺: 922.34.

### Example 6: Photoresist Composition Comprising Compound G

(1) matrix:

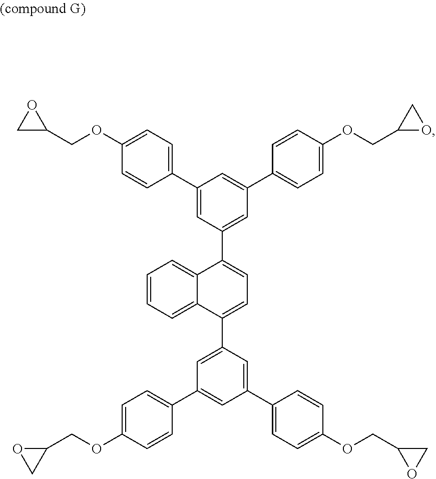 [SMILES: c1ccc2c(-c3cc(-c4ccc(OCC5CO5)cc4)cc(-c4ccc(OCC5CO5)cc4)c3)ccc(-c3cc(-c4ccc(OCC5CO5)cc4)cc(-c4ccc(OCC5CO5)cc4)c3)c2c1]

50 mg;
(2) photoacid generator: benzyl(4-hydroxyphenyl)methylsulfonium hexafluoroantimonate, 2.5 mg; and
(3) organic solvent: propylene glycol methyl ether acetate (PGMEA), 2 mL.

Among them, the compound G was prepared by a method similar to that of Example 1 or 2. HRMS(MALDI) [M+H]⁺: 872.32.

### Test Example 1

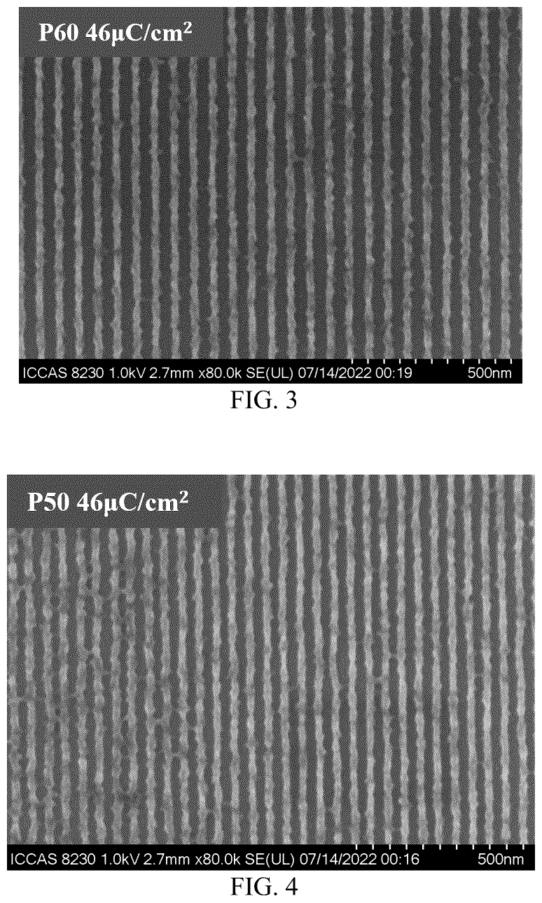

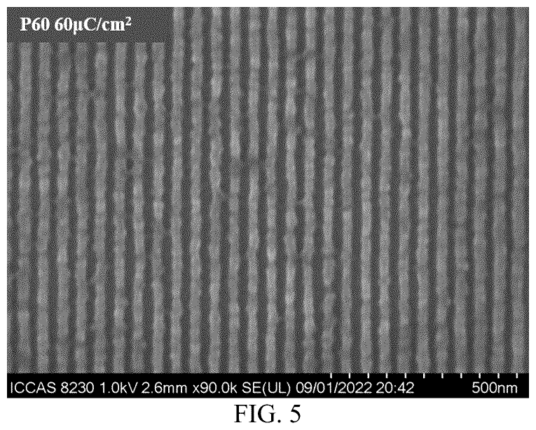

The photoresist composition comprising compound C in Example 3 was adopted, and a 50-100 nm photoresist film was obtained by spin coating a silicon wafer. The photoresist composition had good film-forming performance, and the obtained film had uniform thickness. The electron beam lithography was performed in the National Center for Nanoscience and Technology, and lithographic patterns with line widths of 30 nm and 25 nm were obtained at a dosage of 46 μC/cm², as shown in FIGS. 3 and 4. The patterns were used as negative photoresists, which have high resolution and high sensitivity.

### Test Example 2

The photoresist composition comprising compound F in Example 4 was adopted, and a 50-100 nm photoresist film was obtained by spin coating a silicon wafer. The photoresist composition had good film-forming performance, and the obtained film had uniform thickness. The electron beam lithography was performed in the National Center for Nanoscience and Technology, and a lithographic pattern with a line width of 30 nm was obtained, as shown in FIG. 5. The pattern was used as a negative photoresist, which has high resolution and high sensitivity.

The embodiments of the technical solutions of the present disclosure have been described above by way of example. It should be understood that the protection scope of the present disclosure is not limited to the embodiments described above. Any modification, equivalent replacement, improvement, and the like made by those skilled in the art without departing from the spirit and principle of the present disclosure shall fall within the protection scope of the claims of the present application.

1. A compound represented by formula (I): 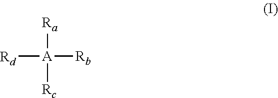
wherein A is selected from a fused ring aromatic hydrocarbon;
Rₐ, R_{b}, R_{c}, and R_{d} are the same or different and are each independently selected from H or 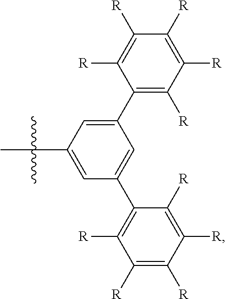
provided that at least one of Rₐ, R_{b}, R_{c}, and R_{d} is 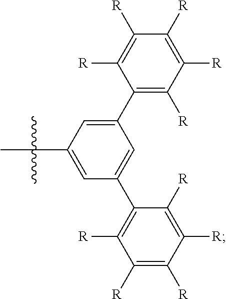
each R is the same or different and is independently selected from H, —OC₁₋₂₀ alkyl, or OR₁, provided that at least one R is OR₁ or H; R₁ is selected from the following groups that are unsubstituted or optionally substituted with one, two, or more R₁₁: C₂₋₂₀ alkenyl and 3- to 20-membered epoxy; each R₁₁ is the same or different and is independently selected from oxo(═O) and the following groups that are unsubstituted or optionally substituted with one, two, or more R₁₂: C₁₋₂₀ alkyl, C₁₋₂₀ alkoxy, C₂₋₂₀ alkenyl, 3- to 20-membered heterocyclyl, and C₆₋₂₀ aryl; each R₁₂ is the same or different and is independently selected from C₁₋₂₀ alkyl, C₂₋₂₀ alkenyl, C₁₋₂₀ alkoxy, and C₆₋₂₀ aryl.

2. The compound according to claim 1, wherein the fused ring aromatic hydrocarbon is selected from C₉₋₄₀ aromatic hydrocarbon, preferably C₁₀₋₁₆ aromatic hydrocarbon;
preferably, the fused ring aromatic hydrocarbon is selected from naphthalene, anthracene, phenanthrene, or pyrene.

3. The compound according to claim 1, wherein Rₐ, R_{b}, R_{c}, and R_{d} are the same or different and are each independently selected from H or 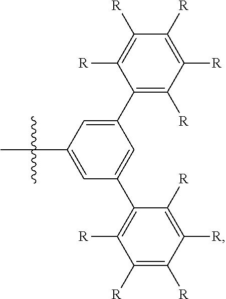
provided that one, two, three, or four of Rₐ, R_{b}, R_{c}, and R_{d} are 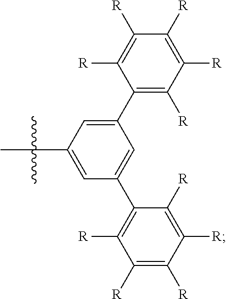
preferably, Rₐ, R_{b}, R_{c}, and R_{d} are selected from 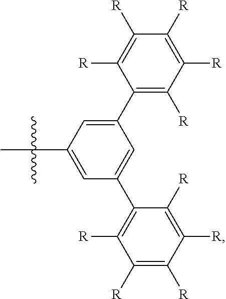
wherein in the 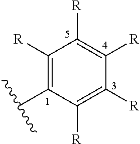
group, when there is only one R, it is preferably linked at position 4; when there are two R, they are preferably linked at positions 3 and 4, or positions 4 and 5; when there are three R, they are preferably linked at positions 3, 4, and 5;
preferably, each R is the same or different and is independently selected from H or OR₁, provided that R is not all H; R₁ is selected from the following groups that are unsubstituted or optionally substituted with one, two, or more R₁₁: C₁₋₆ alkyl and 3- to 8-membered heterocyclyl; each R₁₁ is the same or different and is independently selected from oxo(═O) and the following groups that are unsubstituted or optionally substituted with one, two, or more R₁₂: C₁₋₆ alkyl, C₂₋₆ alkenyl, and oxygen-containing 3- to 8-membered heterocyclyl;
preferably, each R is the same or different and is independently selected from H or OR₁, provided that R is not all H; R₁ is selected from the following groups that are unsubstituted or optionally substituted with one, two, or more R₁₁: oxygen-containing 3- to 8-membered heterocyclyl, C₂₋₆ alkenyl-C₁₋₆ alkyl, and oxygen-containing 3- to 8-membered heterocyclyl-C₁₋₆ alkyl; each R₁₁ is the same or different and is independently selected from oxo(═O) and C₁₋₆ alkyl;
preferably, each R is the same or different and is independently selected from H or OR₁, provided that R is not all H; R₁ is selected from 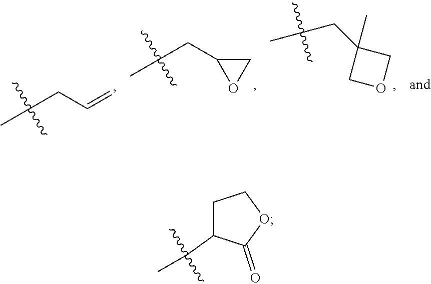
the “ [symbol] ” is a linking site.

4. The compound according to claim 1, wherein the compound represented by formula (I) preferably has a structure represented by formula (A) or formula (B): 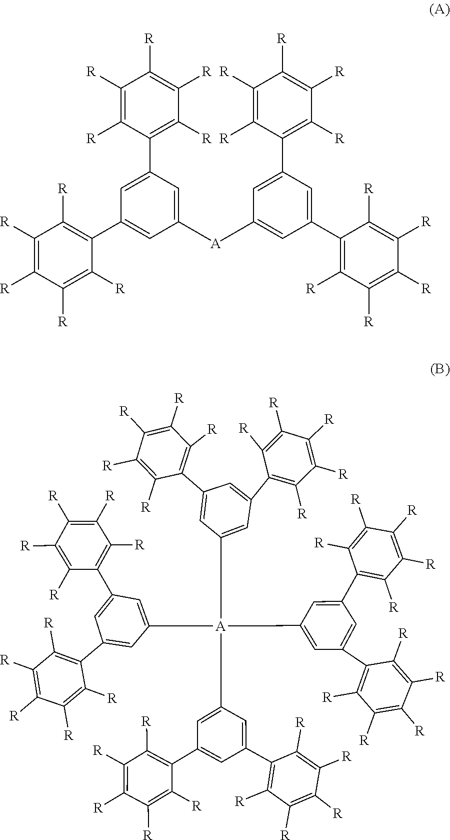
wherein R and A have the definitions described in claim 1.

5. The compound according to claim 1, wherein the compound is selected from the following structures:

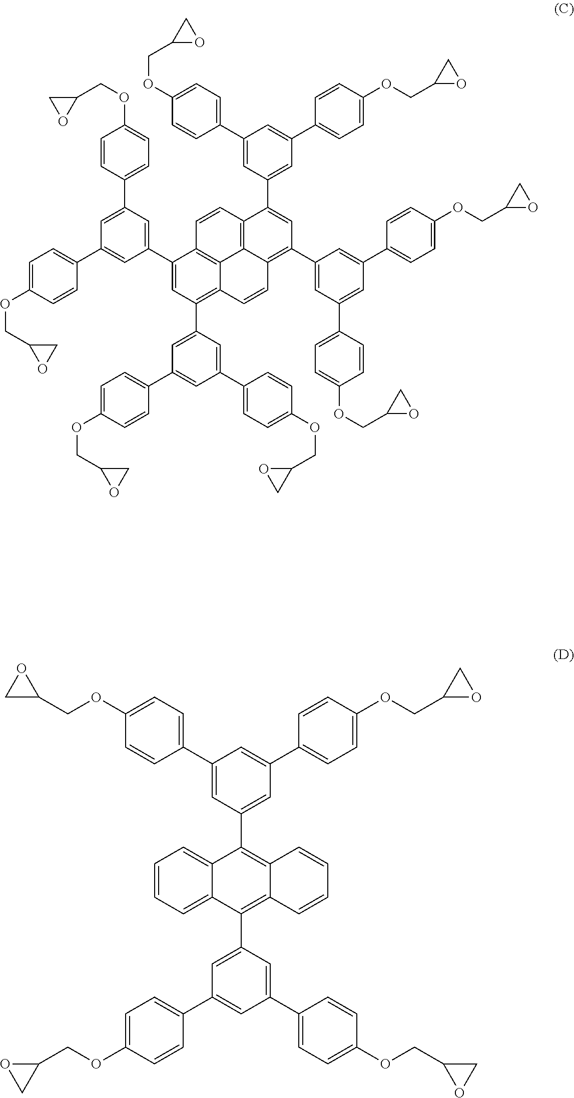 [SMILES: c1cc(-c2cc(-c3ccc(OCC4CO4)cc3)cc(-c3cc(-c4cc(-c5ccc(OCC6CO6)cc5)cc(-c5ccc(OCC6CO6)cc5)c4)c4ccc5c(-c6cc(-c7ccc(OCC8CO8)cc7)cc(-c7ccc(OCC8CO8)cc7)c6)cc(-c6cc(-c7ccc(OCC8CO8)cc7)cc(-c7ccc(OCC8CO8)cc7)c6)c6ccc3c4c65)c2)ccc1OCC1CO1 ; c1ccc2c(-c3cc(-c4ccc(OCC5CO5)cc4)cc(-c4ccc(OCC5CO5)cc4)c3)c3ccccc3c(-c3cc(-c4ccc(OCC5CO5)cc4)cc(-c4ccc(OCC5CO5)cc4)c3)c2c1]

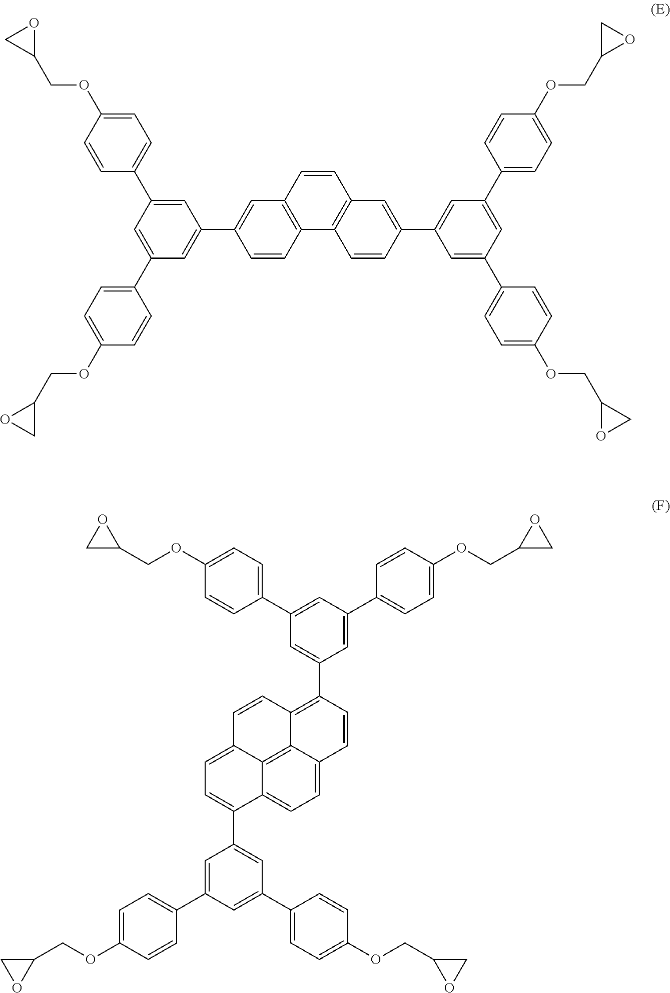 [SMILES: c1cc(-c2cc(-c3ccc(OCC4CO4)cc3)cc(-c3ccc4c(ccc5cc(-c6cc(-c7ccc(OCC8CO8)cc7)cc(-c7ccc(OCC8CO8)cc7)c6)ccc54)c3)c2)ccc1OCC1CO1 ; c1cc(-c2cc(-c3ccc(OCC4CO4)cc3)cc(-c3ccc4ccc5c(-c6cc(-c7ccc(OCC8CO8)cc7)cc(-c7ccc(OCC8CO8)cc7)c6)ccc6ccc3c4c65)c2)ccc1OCC1CO1]

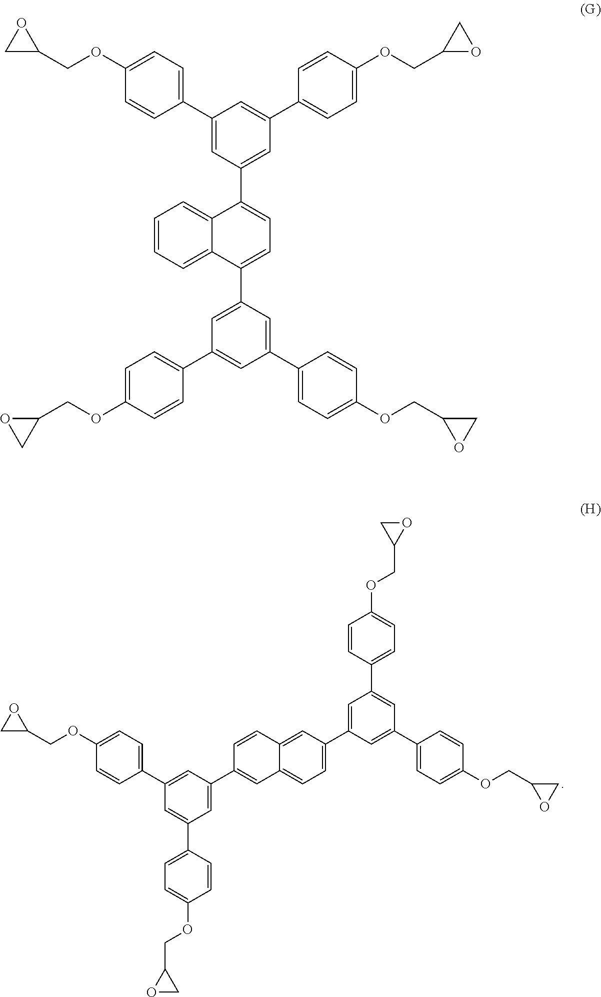 [SMILES: c1ccc2c(-c3cc(-c4ccc(OCC5CO5)cc4)cc(-c4ccc(OCC5CO5)cc4)c3)ccc(-c3cc(-c4ccc(OCC5CO5)cc4)cc(-c4ccc(OCC5CO5)cc4)c3)c2c1 ; c1cc(-c2cc(-c3ccc(OCC4CO4)cc3)cc(-c3ccc4cc(-c5cc(-c6ccc(OCC7CO7)cc6)cc(-c6ccc(OCC7CO7)cc6)c5)ccc4c3)c2)ccc1OCC1CO1]

6. A preparation method for the compound according to claim 1, comprising the following steps: 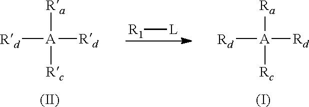
reacting a compound represented by formula (II) with R₁-L to give the compound represented by formula (I);
wherein A, Rₐ, R_{b}, R_{c}, R_{d}, and R₁ have the definitions described in claim 1;
R′ₐ, R′_{b}, R′_{c}, and R′_{d} are 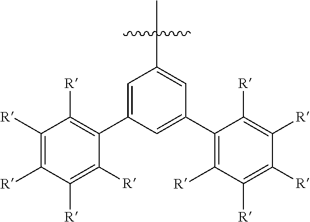
or H, provided that not all are H; each R′ is the same or different and is independently selected from OH or H, provided that not all are H; L is selected from a leaving group such as halogen or p-toluenesulfonate.

7. Use of the compound according to claim 1 in lithography, such as use thereof in a photoresist, preferably use thereof in the preparation of a negative photoresist.

8. A negative photoresist composition, comprising a matrix, wherein the matrix is selected from at least one of compounds represented by formula (I);
preferably, the composition further comprises a photoacid generator, wherein the photoacid generator is, for example, selected from ionic or non-ionic acid generators, such as at least one of triphenyl sulfonium triflate, triphenylsulfonium nonaflate, bis(4-tert-butylphenyl)iodonium p-toluenesulfonate, N-hydroxynaphthalimide triflate, and benzyl(4-hydroxyphenyl)methylsulfonium hexafluoroantimonate;
preferably, the composition further comprises an organic solvent, wherein the organic solvent is, for example, selected from alkane, ester, ether, and haloalkane compounds; the preferred organic solvent is at least one of 1,2,3-trichloropropane, anisole, propylene glycol methyl ether acetate, propylene glycol monoacetate, propylene glycol diacetate, ethyl lactate, propylene glycol monomethyl ether, methyl ethyl ketone, methyl isobutyl ketone, neopentyl acetate, butyl acetate, diethylene glycol ethyl ether, dichloromethane, and tetrahydrofuran.

9. The negative photoresist composition according to claim 8, wherein in the photoresist composition, the mass of the matrix accounts for 2%-30%, preferably 4%-20%, of the total mass of the negative photoresist composition;
preferably, in the photoresist composition, the mass of the photoacid generator accounts for 2%-30%, preferably 5%-20%, of the mass of the matrix;
preferably, in the photoresist composition, the mass of the organic solvent accounts for 70%-96% of the total mass of the photoresist.

10. Use of the compound according to claim 1 in ultraviolet (365 nm) lithography, deep ultraviolet (248 nm and 193 nm) lithography, extreme ultraviolet (13.5 nm, EUV) lithography, and electron beam lithography (EBL).

11. Use of the compound according to claim 8 in ultraviolet (365 nm) lithography, deep ultraviolet (248 nm and 193 nm) lithography, extreme ultraviolet (13.5 nm, EUV) lithography, and electron beam lithography (EBL).
